(19) United States
(12) Patent Application Publication
(10) Pub. No.: US 2026/0318794 A1
(43) Pub. Date: Oct. 15, 2026

(54) SYSTEMS, DEVICES AND METHODS FOR SAMPLE ENRICHMENT

(71) Applicant: The Regents of the University of California, Oakland, CA (US)

(72) Inventors: Yu-Hwa Lo, San Diego, CA (US); Edward Wang, San Diego, CA (US)

(21) Appl. No.: 19/482,690

(22) PCT Filed: May 9, 2024
(86) PCT No.: PCT/US2024/028655

§ 371 Date: Nov. 7, 2025

Related U.S. Application Data
(60) Provisional application No. 63/501,025, filed on May 9, 2023.

(51) Int. Cl. G01N 1/40; C12Q 1/6806; G01N 1/44

(52) U.S. Cl. CPC G01N 1/40; C12Q 1/6806; G01N 1/4005; G01N 1/44

(57) ABSTRACT

Disclosed are devices, systems and methods for enhanced sample enrichment. In some aspects, a sample preconcentration technique includes airjet-induced liquid phase enrichment. In other aspects, a sample preconcentration technique includes liquid phase enrichment through an evaporative gradient induced by passive or active airflow.

### CROSS-REFERENCE TO RELATED APPLICATIONS

This patent document claims the benefits and priority of U.S. Provisional Patent Application No. 63/501,025, titled “SYSTEMS, DEVICES AND METHODS FOR SAMPLE ENRICHMENT” and filed on May 9, 2023. The entire contents of the aforementioned patent application are incorporated by reference as part of the disclosure of this application.

### STATEMENT REGARDING FEDERALLY SPONSORED RESEARCH OR DEVELOPMENT

This invention was made with government support under grant number 2020-20090400002 awarded by Intelligence Advanced Research Projects Activity (IARPA). The government has certain rights in the invention.

### TECHNICAL FIELD

This patent document relates to chemical and biological sample preparation technologies.

### BACKGROUND

Polymerase chain reaction (PCR) is a widely used method for amplification of nucleic acid targets in samples for various biological assays. PCR is commonly used in molecular biology to make copies of a particular region of nucleic acids, such as deoxyribonucleic acid (DNA), starting from a few copies up to several orders of magnitude. It involves a series of cycles involving different temperature points to facilitate DNA melting and enzymatic replication.

For biological assays based on an amplification process, such as PCR or Loop-mediated isothermal amplification (LAMP), the amplification process is capable of detecting single copies of the target molecules. However, false negatives can occur due to loss during sample preparation. Target loss in conventional amplification-based assays has become a serious problem, having loss up to about 99% of the target molecule. Conventional sample preparation steps, such as capture methods using beads, can cause about 99% of targets in the original sample to never get to a PCR tube by the end of sample preparation.

What is needed is sample enrichment techniques that preconcentrate a sample before the characterization technique is implemented.

### SUMMARY

Disclosed are devices, systems and methods for enhanced sample enrichment. In some aspects, a sample preconcentration technique is described through airjet-induced liquid phase enrichment.

In some aspects, a disclosed method for sample enrichment comprises distributing a plurality of particles in a solution under ambient evaporation and applying a flow of gas through one or more airjets to increase concentration of the plurality of particles in the solution.

In some aspects, a disclosed method for sample enrichment comprises distributing a plurality of particles in a solution under ambient evaporation, applying the solution to a porous membrane, and applying a flow of gas through one or more airjets to increase concentration of the plurality of particles in the solution.

In some aspects, a disclosed method for sample enrichment comprises distributing a plurality of particles in a solution under ambient evaporation, applying the solution to a membrane, the membrane comprising a first porous material and a second porous or non-porous material (i.e., a material that is at least less porous material than the first porous material), and applying airflow from one or more sources to increase concentration of the plurality of particles in the solution.

In some aspects, a method for sample enrichment comprises distributing a plurality of particles in a solution under ambient evaporation, applying the solution to a membrane, the membrane comprising a first porous material and a second porous or non-porous material (i.e., a material that is at least less porous material than the first porous material), and applying a differential evaporation rate through airflow from one or more sources between one or more locations of the membrane to increase the concentration of the plurality of particles or dissolved or suspended components in the solution at locations on the membrane where the evaporation rate of the solution is greater.

In some aspects, a disclosed device for sample enrichment comprises one or more sources of airflow, a substrate (wherein the substrate may include a non-porous material, and wherein the substrate includes a base and an exterior wall), a membrane (wherein the membrane comprises a first porous material and a second porous or non-porous material (i.e., a material that is at least less porous material than the first porous material), and wherein the first porous material is thinner than the second less porous or non-porous material), and one or more heating units (wherein the one or more heating units are attached to the substrate, the one or more sources of airflow, or a combination thereof).

In some embodiments, the flow of gas applied through one or more airjets reverses radial flow by establishing a large evaporative flux at the center of a droplet of the solution. In some embodiments, the flow of gas applied through one or more airjets displaces a boundary layer comprised of saturated water vapor which lies directly above a liquid-air interface. In some embodiments, displacement of the boundary layer leads to enhanced diffusion across the liquid-air interface and shifts an evaporative gradient towards an area under the one or more airjets. In some embodiments, the droplet is a sessile droplet.

In some embodiments, the method of sample enrichment further comprises controlling an overall evaporation rate based at least on controlling temperature of the flow of gas through the one or more airjets. In some embodiments, the method of sample enrichment further comprises controlling an overall evaporation rate based at least on controlling temperature of the solution.

In some embodiments, the flow of gas applied through the one or more airjets includes positioning a nozzle above the droplet and directing a jet of air perpendicular to its surface.

In some embodiments, the plurality of particles include at least one of nucleic acid molecules, polynucleotides, viruses, dissolved components, suspended components, or other type of chemical molecules, compounds, or nanoscale or microscale structures, or a combination thereof.

In some embodiments, the gas includes at least one of nitrogen, argon, air, or other gas, or a combination thereof.

In some embodiments, the method of sample enrichment further comprises adding a surfactant to the solution before applying the flow of gas through the one or more airjets to increase concentration of the plurality of particles in the solution. In some embodiments, the surfactant is methyl cellulose, polydimethylsiloxane, or sodium dodecyl sulfate.

In some embodiments, the one or more airjets are set up in parallel. In some embodiments, the one or more airjets are set up in an array. In some embodiments, the one or more airjets are aimed at the same location. In some embodiments, the one or more airjets are aimed at more than one location.

In some embodiments, the porous material(s) of the membrane includes at least one of paper, cotton, microfiber, bamboo, nitrocellulose, or other porous material, or a combination thereof. In some embodiments, the porous membrane is a cellulose-based material. In some embodiments, the first porous material is thinner than the second less porous or non-porous material.

In some embodiments, the one or more sources of airflow over one or more areas of the membrane are passive. In some embodiments, the one or more sources of airflow over one or more areas of the membrane are active.

In some aspects, the disclosure provides for a device or system for sample enrichment comprising an airjet-induced liquid phase enrichment technique.

In some aspects, the disclosed device comprises one or more airjets, a membrane, and a heating unit. In other aspects, the disclosed device comprises one or more sources of airflow, a membrane, and a heating unit. The heating unit may be, but is not limited to, a metal coil and heating pad. In some embodiments, the airjets may additionally comprise a heating unit. The heating unit may, in some embodiments, be a metal coil contained within each of the one or more airjets.

In some embodiments, the membrane is porous. In some embodiments, the membrane comprises a first porous material and a second porous or non-porous material (i.e., a material that is at least less porous material than the first porous material). In some embodiments, the membrane includes at least one of paper, cotton, microfiber, bamboo, nitrocellulose, or other material, or a combination thereof. In some embodiments, the porous material(s) includes one or more of paper, cotton, microfiber, bamboo, nitrocellulose, or other porous material, or a combination thereof.

In some embodiments, the one or more airjets are set up in parallel. In some embodiments, the one or more airjets are set up in an array. In some embodiments, the one or more airjets are aimed at the same location. In some embodiments, the one or more airjets are aimed at more than one location.

In some embodiments, the one or more sources of airflow are set up in parallel. In some embodiments, the one or more sources of airflow are set up in an array. In some embodiments, the one or more sources of airflow are aimed at the same location. In some embodiments, the one or more sources of airflow are aimed at more than one location.

In some embodiments, the one or more sources of airflow are passive. In some embodiments, the one or more sources of airflow are active.

The subject matter described in this patent document can be implemented in specific ways that provide one or more of the following features.

### BRIEF DESCRIPTION OF THE DRAWINGS

FIG. 1 shows a diagram of an example embodiment of a sample enrichment method in accordance with the present technology.

FIG. 2 shows a diagram of an example passive airflow sample enrichment system, in accordance with the present technology.

FIG. 3 shows a diagram depicting a cross-section of a passive airflow sample enrichment system, in accordance with the present technology.

FIG. 4 shows a schematic of airjet enrichment to reverse coffee-ring flows and the comparison of the resulting deposition patterns with that of ambient evaporation.

FIG. 5 shows: (a) a diagram and images of fluid behavior under an example embodiment of an airjet, in accordance with the present technology, in the absence (−M.C.) and presence (+M.C.) of the methyl cellulose surfactant in a droplet as the process progresses (from top to bottom), where the scale bar is 4 mm; and (b) a diagram and images from a 96-well plate after enrichment with (+M.C.) and without (−M.C.) methyl cellulose.

FIG. 6 shows an image showing an example implementation of enrichment of rhodamine in ethanol.

FIG. 7 shows fluorescent images of example results from an enrichment implementation in accordance with the present technology, before and after 5 minutes of enrichment with equal exposure time for FITC-tagged DNA oligonucleotides, green fluorescent protein (GFP), 20 nm streptavidin-functionalized quantum dots, and 1 μm fluorescent beads.

FIG. 8 shows images depicting a comparison of duplicate ELISA wells for direct and enriched samples after 3 minutes of Tetramethylbenzidine (TMB) development.

FIG. 9A shows a diagram illustrating an exemplary heated airjet (left) and associated thermal images (right) taken at specified voltages applied to the heating element.

FIG. 9B shows data plots depicting temperature profiles of the liquid surface for different voltages and airflow speeds in example implementations of the heated airjet.

FIG. 10 shows an image of an example embodiment of the heated airjet used for enhanced sample enrichment, in accordance with the present technology.

FIG. 11 shows a data plot depicting average temperature vs. applied voltage for the pipette's exit nozzle for an example implementation of the sample enrichment technique using the example heated airjet of FIG. 10.

FIG. 12A shows a diagram illustrating an example embodiment of a double-stage enrichment process, in accordance with the present technology.

FIG. 12B shows exemplary data from example implementations of the double-stage enrichment process of FIG. 12A.

FIG. 13 shows data plots depicting a comparison of cumulative ΔCq (orange) and fold-enrichment (green) for λ-DNA, HeLa-S3 RNA, and heat-inactivated SARS-COV-2 in 0.1 mM EDTA for solutions with PEG (+PEG) (left plot) and without PEG (−PEG) (right plot).

FIG. 14 shows a data plot depicting the Cq vs. EDTA concentration for a solution of λ-DNA in an example implementation of the disclosed technology.

### DETAILED DESCRIPTION

While the present disclosure is capable of being embodied in various forms, the description below of several embodiments is made with the understanding that the present disclosure is to be considered as an exemplification of the invention and is not intended to limit the invention to the specific embodiments illustrated.

Headings are provided for convenience only and are not to be construed to limit the invention in any manner. Embodiments illustrated under any heading may be combined with embodiments illustrated under any other heading.

To the extent any materials incorporated herein by reference conflict with the present disclosure, the present disclosure controls.

### Sample Enrichment

For nucleic acid (e.g., DNA and RNA) assays based on an amplification process (e.g., qPCR, RT-PCR, LAMP, etc.), the amplification process itself is highly sensitive and capable of detecting single copies of the target molecules. However, if the target chemical species in a sample are lost during sample preparation, the test result could become a false negative result, where the sensitivity of the test may have been compromised.

Presently, the potential for target loss in conventional amplification-based assays has become a serious problem because current methods for sample preparation (e.g., using magnetic beads or any target capture methods) have been shown to lose about 99% of the targets. For example, a typical elution process to elute nucleic acids from magnetic beads uses a sample volume of about 150 μL within which the target nucleic acids to be extracted are. However, the typical elution process only uses 2 μL of the elution buffer for adding to a 20 μL PCR mix. This means only 1-2% of target nucleic acids are added to the PCR mix from the last step alone, without considering all the previous steps for target capture by beads, bead collection, washing, etc., which may result in substantial target loss prior to the elution process These conventional sample preparation steps create the problem that more than 99% of targets in the original sample likely never get to the PCR tube by the end of sample preparation.

As an illustrative example, a nasal swap sample for a Covid test may collect around 100 viral particles (i.e., with 50 copies of genetic RNA). As such, one would have a very high chance (e.g., 50% chance) of producing a false negative result in conventional practice, even through PCR or LAMP process itself can be highly sensitive.

What is needed is sample enrichment techniques that preconcentrate a sample before the characterization technique is implemented.

Disclosed are devices, systems and methods for enhanced sample enrichment. In some aspects, a sample preconcentration technique is described through airjet-induced liquid phase enrichment. In some aspects, a sample preconcentration technique includes liquid phase enrichment through an evaporative gradient induced by passive or active airflow.

The disclosed technology can offer an effective solution for present sample collection challenges with target loss and sample bottleneck. In some implementations of the disclosed technology, for example, example embodiments of the disclosed sample enrichment devices, systems, and methods can collect>60% target molecules from the original sample, representing 10× to 100× performance enhancement compared to the current state of the art, which can be translated to nearly 0% false negative by the disclosed sample enrichment devices, systems, and methods. For other tests of low abundant targets (e.g., cell-free DNAs in serum), the disclosed sample enrichment devices, systems, and methods can greatly improve the sensitivity and reproducibility. For example, when the method in accordance with the disclosed technology is applied to protein or antibody tests (e.g., ELISA) where amplification is not possible, the method can increase the sensitivity of the existing assay by 10× to 100× due to the enhanced target collection efficiency.

In some aspects, the disclosed systems, devices, and methods provide an effective sample enrichment technique that can reverse the “coffee ring effect” (CRE) to retain analytes in the liquid during an accelerated evaporation process, confining the analytes in a well-defined area on the liquid surface for easy collection and transfer, and achieving a certain level of selectivity to minimize co-enrichment of salt and other unwanted species.

CRE refers to the analytes and all other non-volatile species, including salts, precipitating on the dry surface in a spatially-distributed pattern during evaporation. CRE occurs because an evaporating droplet forms a differential evaporative gradient having the highest evaporation rate at the droplet's perimeter. This evaporation rate gradient induces a radially outward flow to drive fluid within the droplet towards the periphery to replenish the lost fluid at the liquid-solid interface. As a result, any solutes or suspended particles are carried to the contact line and deposited. The disclosed technology is configured to solve one bottleneck problem for all molecular diagnosis methods: loss of target molecules during sample preparation. It can significantly increase analyte collection efficiency several hundred times more than current practices thus improving the accuracy and repeatability of in-vitro diagnostic/point-of-care assays.

In some example embodiments, airjet-induced evaporation is used to reverse the coffee-ring flow. The airjet drives analytes from a relatively large droplet periphery towards a smaller central region under an airjet apparatus, in accordance with the disclosed sample enrichment devices, systems, and methods. Coffee-ring flow reversal can be achieved by aiming a nozzle of the airjet to be perpendicular to the liquid surface and initiating the airjet to increase local evaporation. The localized increase in evaporative flux establishes a differential evaporative gradient and induces fluid flows towards the area to replenish the lost liquid. During this process, the flow carries dissolved molecules or suspended particles, which become highly concentrated in a small, localized area underneath the nozzle.

The disclosed sample enrichment devices, systems, and methods have a cost-effective design that can include an air source, an air heater, and an airjet nozzle in some example embodiments. In example implementations of the disclosed sample enrichment devices, systems, and methods, it was found that the presence of surfactant (e.g., methyl cellulose) in the sample can suppress fluid turbulence and improve performance. The effectiveness of the approach was demonstrated by concentrating a range of particles and biological analytes—specifically nucleic acids, proteins, and viral and other particles. Spiked samples containing λ-DNA, HeLa-S3 RNA, and heat-inactivated SARS-COV-2 were used as model analytes and reduction of the cycle threshold value (Cq) in real-time PCR (qPCR) analysis was used to quantify analyte enrichment and collection efficiency.

### Example Embodiments and Implementations of the Disclosed Technology

### Sample Preconcentration Through Airjet-Induced Liquid Phase Enrichment

Sample preparation is essential for nucleic acid amplification assays, affecting their sensitivity and reliability. However, this process often results in a significant loss or dilution of analyte, which becomes a bottleneck that limits downstream assay performance—particularly for assays that accept a limited input sample volume.

To overcome this challenge, the disclosed technology provides an evaporation-based, sample enrichment method that includes using an airjet (in some example embodiments) to concentrate various substances within a small, defined volume by reversing the coffee-ring effect. The concentrated sample can then be collected to increase the initial sample quantity for subsequent analysis. The effectiveness of the reported airjet-based sample enrichment was quantified using qPCR tests for the detection of λ-DNA, HeLa-S3 RNA, and heat-inactivated SARS-COV-2 samples. Comparisons between airjet and conventional evaporative methods demonstrated significant advantages of the disclosed airjet enrichment techniques, including the ability to concentrate a high percentage of analyte within a 1 μL volume. The enhanced sample enrichment method in accordance with the present technology can be integrated and adapted for several fluid volumes commonly found in nucleic acid sample preparation procedures. Here, airjet enrichment can reduce the overall Cq by an average of 9.27 cycles for each analyte, representing a 600-fold enrichment from the initial concentration. In addition, in some example embodiments, to perform selective enrichment of nucleic acid and reduce salt-based interference in downstream analysis, PEG can be added to reduce the co-enrichment of salt. Example studies employing some example embodiments of the sample enrichment technique demonstrated that airjet enrichment technology can be easily integrated into existing laboratory protocols with minimal modification to significantly improve the performance of nucleic acid amplification assays.

The analytical sensitivity in detecting biological analytes is heavily influenced by the upstream sample preparation steps, in addition to the recognition and signal transduction mechanisms that follow. Significant advancements in detection schemes for nucleic acid biosensors have been reported, yet progress in sample enrichment and pre-treatment has been slower. Currently, the use of magnetic beads is a popular method for sample preparation, including nucleic acid extraction and purification. However, a major challenge with this method is the loss and/or dilution of analyte that occurs during the transfer, washing, and elution steps. In fact, elution of purified nucleic acid into a relatively large (typically 50-100 μL) volume of elution buffer for an assay that accepts a small (typically 1-5 μL) sample input volume will result in a low (1-10%) sample transfer efficiency. This efficiency is further reduced by losses in the many intermediate transferring and washing steps before elution. The low efficiency of the sample preparation workflow contributes to the sensitivity bottleneck in current biosensors for in-vitro diagnosis (IVD) and point of care (POC) testing.

To address this challenge, a sample processing method was developed to concentrate the analytes within a small volume (e.g., 1-2 μL) prior to testing, such as nucleic acid amplification reactions. In addition, the exemplary liquid-phase sample enrichment method has some selectivity for nucleic acids or protein to suppress the simultaneous concentration of extraneous solutes and improve signal-to-noise. This method is applicable to various biomolecules, as well as amplification and detection methods.

Many sample preconcentration techniques have been reported using the properties of solid-phase affinity, liquid-phase affinity, and electrophoresis. However, these methods are limited in scope, and have not replaced the prevailing magnetic bead method because of their dependence on the analyte's charge, surface binding affinity, or solvent miscibility. Conceptually, the most straightforward preconcentration method that is analyte independent is evaporation. Because most analytes in biosensors are non-volatile, volume reduction by evaporation increases the analyte concentration. However, this simple approach has shown limited success to date due to the well-known coffee-ring effect (CRE) where the analytes and all other non-volatile species including salts, precipitate on the dry surface in a spatially-distributed pattern during evaporation. While some works claim that the coffee-ring patterns mark the distributions of the analytes that may facilitate resuspension or in-situ detection, the CRE is generally an undesirable effect that compromises sensitivity, reproducibility, and operability for IVD and POC testing, and as a result, significant efforts have been made to overcome the formation of coffee rings and to keep analytes in liquid solution.

The CRE pattern occurs because an evaporating droplet forms a differential evaporative gradient having the highest evaporation rate at the droplet's perimeter. This evaporation rate gradient induces a radially outward flow to drive fluid within the droplet towards the periphery to replenish the lost fluid at the liquid-solid interface. As a result, any solutes or suspended particles are carried to the contact line and deposited. CRE reversal can be achieved through electroosmosis, electrowetting, surface wave acoustics, infrared laser heating, or microfabricated surface patterning. However, these processes are generally complicated and depend on the properties of the particles such as particle size, charge, hydrophobicity, etc. Above all, with or without CRE, evaporation methods enrich the analytes and other solutes, such as salts, that can interfere with downstream analysis.

The example embodiments and implementations described herein for the present technology demonstrates an effective sample enrichment method that can reverse the CRE to retain analytes in the liquid during an accelerated evaporation process; confining the analytes in a well-defined area on the liquid surface for easy collection and transfer by a pipette, and achieve a certain level of selectivity to minimize co-enrichment of salt and other unwanted species.

For some example embodiments in accordance with the present technology, a sample enrichment technique uses airjet-induced evaporation to reverse the coffee-ring flow. For other example embodiments in accordance with the present technology, particles (e.g., chemical species, dissolved or suspended components like molecules or compounds) in solution are set under ambient evaporation and a flow of gas, such as from one or more fans, is applied to increase concentration of the particles in the solution. In other example embodiments, particles in solution are set under ambient evaporation and one or more sources of airflow, such as from one or more fans or one or more vacuums, generate a differential evaporation gradient. In other example embodiments, particles in solution are set under ambient evaporation and one or more sources of passive airflow to generate a differential evaporation gradient. The flow of gas can be generated from airjets, fans, vacuums, and like devices capable of moving gases, or a combination thereof.

For example, for embodiments of the disclosed sample enrichment devices, systems, and methods including an airjet apparatus, the airjet drives analytes from a relatively large droplet periphery towards a smaller central region under the airjet. Coffee-ring flow reversal is achieved by aiming the nozzle perpendicular to the liquid surface and initiating the airjet to increase local evaporation. The localized increase in evaporative flux establishes a differential evaporative gradient and induces fluid flows towards the area to replenish the lost liquid. During this process, the flow carries dissolved molecules or suspended particles, which become highly concentrated in a small, localized area underneath the nozzle. The disclosed sample enrichment devices, systems, and methods have a cost-effective design that can include an air source, an air heater, and an airjet nozzle in some example embodiments. In example implementations of the disclosed sample enrichment devices, systems, and methods, it was found that the presence of surfactant (e.g., methyl cellulose) in the sample can suppress fluid turbulence and improve performance.

In example implementations of the airjet-based enhanced sample enrichment techniques, it was demonstrated that the effectiveness by concentrating a range of particles and biological analytes—specifically nucleic acids, proteins, and viral and other particles. Spiked samples containing λ-DNA, HeLa-S3 RNA, and heat-inactivated SARS-COV-2 were used as model analytes and reduction of the cycle threshold value (Cq) in real-time PCR (qPCR) analysis was used to quantify analyte enrichment and collection efficiency. To reduce the transfer of salt to downstream reactions, polyethylene glycol (PEG) was added that formed a salt-trapping matrix as evaporation progressed. The airjet enrichment method was employed in both microfluidic and mesofluidic systems to handle sample volumes from <100 μL to >1 mL to address the sample volumes typically used in laboratory PCR testing. The approach addresses a bottleneck in sample preparation by significantly increasing the analyte collection efficiency several hundred times more than current practices. This increase in starting material can improve the accuracy and repeatability of IVD assays.

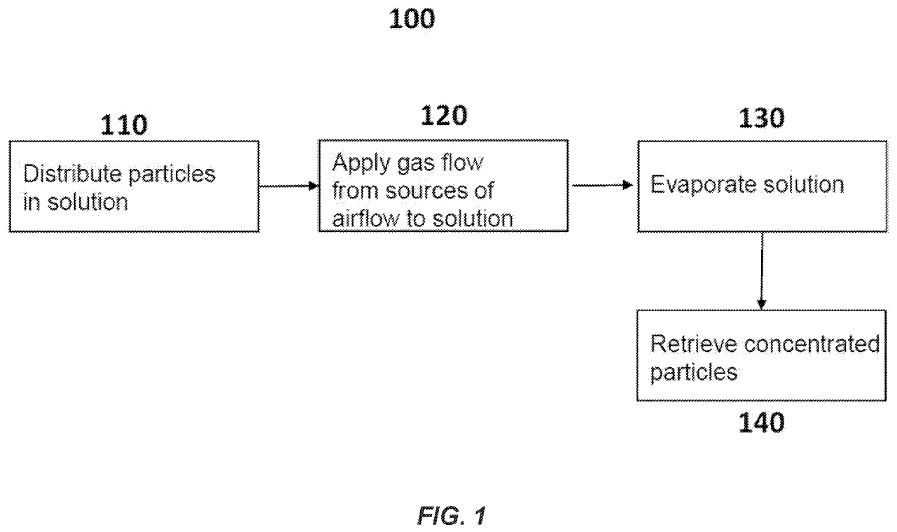

FIG. 1 shows a diagram depicting an example embodiment of a method for sample enrichment in accordance with the present technology, labeled method 100. The method 100 includes a process 110 to distribute a plurality of particles in a solution on a substrate under ambient evaporation. For example, in implementations of the process 110, the particles in the solution can include, but are not limited to, dissolved or suspended components, such as molecules or compounds or nanoscale or microscale structures. The solvent for the solution can be, but is not limited to, water, ethanol, or a combination thereof. The method 100 includes a process 120 to apply a gas flow from one or more sources of airflow to the solution. In some example embodiments of the method 100, the process 120 includes applying the gas flow from one or more airjets to the solution, e.g., directly at the solution or indirectly at the solution. In some example embodiments of the method 100, the process 120 includes applying the gas flow through one or more fans to the solution, e.g., directly or indirectly at the solution. In some example embodiments of the method 100, the process 120 includes a subprocess to selectively position the apparatus to apply the gas flow (e.g., one or more airjets, one or more fans, one or more vacuums, a combination thereof or other air flow apparatus) to create an airflow distribution with predetermined parameters, including flow rate, temperature, gas constituents, etc. Through implementation of the process 120, the method 100 includes a process 130 to controllably evaporate the solution, i.e., area-selective evaporation, and thereby concentrate the particles without loss or harm or other detriment to the particles. Implementations of the process 130 can locate the concentrated particles in a selective portion or portions of the substrate. In some embodiments in accordance with the present technology, the concentrated particles may be moved to one or more locations on the substrate. The present technology can be utilized to differentially split and separate distinct particles dissolved in the solution. The method 100 includes a process 140 to retrieve concentration particles from the substrate. In some embodiments, the concentrated particles are collected from a membrane.

In some embodiments in accordance with the present technology, a method for sample enrichment comprises distributing a plurality of particles in a solution under ambient evaporation and applying a flow of gas through one or more airjets to increase concentration of the plurality of particles in the solution.

In some embodiments in accordance with the present technology, a method for sample enrichment comprises distributing a plurality of particles in a solution under ambient evaporation, applying the solution to a porous membrane, and applying a flow of gas through one or more airjets to increase concentration of the plurality of particles in the solution.

In some embodiments in accordance with the present technology, a method for sample enrichment comprises distributing a plurality of particles in a solution under ambient evaporation, applying the solution to a membrane, the membrane comprising a first material that is porous and a second material that is less porous than the first porous material or non-porous, and applying airflow from one or more sources to increase concentration of the plurality of particles in the solution.

In some embodiments in accordance with the present technology, a method for sample enrichment comprises distributing a plurality of particles in a solution under ambient evaporation, applying the solution to a membrane, the membrane comprising a first material that is porous and a second material that is less porous than the first porous material or non-porous, and applying a differential evaporation rate through airflow from one or more sources between one or more locations of the membrane to increase the concentration of the plurality of particles or dissolved or suspended components in the solution at locations on the membrane where the evaporation rate of the solution is greater. The differential evaporation rate through airflow from one or more sources between one or more locations of the membrane provides regions with evaporation rates greater than those at other locations on the membrane. For example, the airflow from one or more sources can generate a greater evaporation rate at the porous section of the material (e.g., first material) than the evaporation rate at the less porous or non-porous section of the material (e.g., second material), where the evaporation rate may be lower by up to several orders of magnitude.

In some embodiments, the flow of gas applied through one or more airjets reverses radial flow by establishing a large evaporative flux at the center of a droplet of the solution. In some embodiments, the flow of gas applied through one or more airjets displaces a boundary layer comprised of saturated water vapor which lies directly above a liquid-air interface. In some embodiments, displacement of the boundary layer leads to enhanced diffusion across the liquid-air interface and shifts an evaporative gradient towards an area under the one or more airjets. In some embodiments, the droplet is a sessile droplet.

In some embodiments, each nozzle of the one or more airjets is perpendicular to the surface of the solution. In some embodiments, each nozzle of the one or more airjets is set up at an angle to the surface of the solution. In some embodiments, each nozzle of the one or more airjets is set up at an angle to the surface of the solution ranging from about 1 degree, about 2 degrees, about 3 degrees, about 4 degrees, about 5 degrees, about 6 degrees, about 8 degrees, about 10 degrees, about 12 degrees, about 15 degrees, about 20 degrees, about 25 degrees, about 30 degrees, about 35 degrees, about 40 degrees, and about 45 degrees. For example, in some embodiments, each nozzle of the one or more airjets is set up at an angle to the surface of the solution between about 1 degree to about 5 degrees.

In some embodiments, each of the one or more sources of airflow is perpendicular to the surface of the solution. In some embodiments, each of the one or more sources of airflow is set up at an angle to the surface of the solution. In some embodiments, each of the one or more sources of airflow is set up at an angle to the surface of the solution ranging from about 1 degree, about 2 degrees, about 3 degrees, about 4 degrees, about 5 degrees, about 6 degrees, about 8 degrees, about 10 degrees, about 12 degrees, about 15 degrees, about 20 degrees, about 25 degrees, about 30 degrees, about 35 degrees, about 40 degrees, and about 45 degrees. For example, in some embodiments, each of the one or more sources of airflow is set up at an angle to the surface of the solution between about 1 degree to about 5 degrees.

In some embodiments, the method of sample enrichment further comprises controlling an overall evaporation rate based at least on controlling temperature of the flow of gas through the one or more airjets. In some embodiments, the method of sample enrichment further comprises controlling an overall evaporation rate based at least on controlling temperature of the solution.

In some embodiments, the flow of gas applied through the one or more airjets includes positioning a nozzle above the droplet and directing a jet of air perpendicular to its surface.

In some embodiments, the solution is contained within a substrate. In some embodiments, the substrate is flat. In some embodiments, the substrate is convex. In some embodiments, the substrate is concave. In some embodiments, the substrate comprises an exterior wall and a base. In some embodiments, the substrate comprises an exterior wall, one or more interior walls, and a base. In some embodiments, the substrate comprises a non-porous material or porous material that is substantially less porous than the membrane. In some embodiments, the non-porous material of the substrate includes at least one of plastic, metal, glass, or ceramic.

In some embodiments, the solution includes, but is not limited to, ethanol, water, or a combination thereof.

In some embodiments, the particles include, but are not limited to, at least one of nucleic acid molecules, polynucleotides, viruses, dissolved components, suspended components, or a combination thereof. The dissolved components and the suspended components may be compounds such as but not limited to EDTA, dyes, fluorescent compounds, or PEG.

In some embodiments, the gas includes at least one of nitrogen, argon, air, or other gas, or a combination thereof. Gases with a lower moisture content, such as nitrogen and argon, can provide more consistent enrichment results than those with a higher moisture content, such as compressed ambient air.

In some embodiments, the method of sample enrichment further comprises adding a surfactant to the solution before applying the flow of gas through the one or more airjets to increase concentration of the plurality of particles in the solution. In some embodiments, the surfactant is methyl cellulose, polydimethylsiloxane, or sodium dodecyl sulfate.

In some embodiments, the one or more airjets are set up in parallel. In some embodiments, the one or more airjets are set up in an array. In some embodiments, the one or more airjets are aimed at the same location. In some embodiments, the one or more airjets are aimed at more than one location.

In some embodiments, the porous membrane includes at least one of paper, cotton, microfiber, bamboo, nitrocellulose, or other porous material, or a combination thereof. In some embodiments, the porous membrane is a cellulose-based material. In some embodiments, the porous material is thinner than the non-porous or less porous material.

In some embodiments, the one or more sources of airflow over one or more areas of the membrane are passive. In some embodiments, the one or more sources of airflow over one or more areas of the membrane are active.

In some aspects, the disclosure provides for a device or system for sample enrichment comprising an airjet-induced liquid phase enrichment technique.

In some embodiments in accordance with the present technology, a device comprises one or more airjets, a membrane, and a heating unit. In other aspects, the disclosed device comprises one or more sources of airflow, a membrane, and a heating unit. The heating unit may be, but is not limited to, a metal coil and heating pad. In some embodiments, the airjets may additionally comprise a heating unit. The heating unit may, in some embodiments, be a metal coil contained within each of the one or more airjets.

In some embodiments, the membrane is porous. In some embodiments, the membrane comprises a first material that is a porous material and a second material that is a non-porous material or substantially less porous material. In some embodiments, the membrane includes, but is not limited to, paper, cotton, microfiber, bamboo, nitrocellulose, or a combination thereof. In some embodiments, the porous material includes at least one of paper, cotton, microfiber, bamboo, nitrocellulose, or other porous material, or a combination thereof.

In some embodiments, the porous membrane can have pores that range from less than about 1 mm, less than about 0.1 mm, less than about 0.01 mm, less than about 1 μm, less than about 0.5 μm, less than about 0.1 μm, and less than about 0.05 μm. For example, the porous membrane may have pores that are less than about 0.5 μm to less than about 0.1 μm. In some embodiments, the pores in the porous membrane are evenly distributed. In some embodiments, the pores in the porous membrane are randomly distributed.

In some embodiments, the porous material has pores that range from less than about 1 mm, less than about 0.1 mm, less than about 0.01 mm, less than about 1 μm, less than about 0.5 μm, less than about 0.1 μm, and less than about 0.05 μm. For example, the porous material may have pores that are less than about 0.5 μm to less than about 0.1 μm. In some embodiments, the pores in the porous material are evenly distributed. In some embodiments, the pores in the porous material are randomly distributed.

In some embodiments, the porous material has pores that range from about 10 cm, about 5 cm, about 1 cm, about 50 mm, about 10 mm, about 5 mm, about 1 mm, about 0.5 mm, and about 0.1 mm. For example, the non-porous material may range in thickness from about 5 cm to about 0.5 mm.

In some embodiments, the membrane can conduct heat. In some embodiments, the porous material can conduct heat. In some embodiments, the non-porous material or substantially less porous material can conduct heat.

In some embodiments, the one or more airjets are set up in parallel. In some embodiments, the one or more airjets are set up in an array. In some embodiments, the one or more airjets are aimed at the same location. In some embodiments, the one or more airjets are aimed at more than one location.

In some embodiments, the one or more sources of airflow are set up in parallel. In some embodiments, the one or more sources of airflow are set up in an array. In some embodiments, the one or more sources of airflow are aimed at the same location. In some embodiments, the one or more sources of airflow are aimed at more than one location.

In some embodiments, the one or more sources of airflow are passive. In some embodiments, the one or more sources of airflow are active. An example of an active source of airflow may be, but is not limited to, fans.

In some aspects, a disclosed device for sample enrichment comprises one or more sources of airflow; a substrate, wherein the substrate is a non-porous material (or material of substantially low porosity), and wherein the substrate includes a base and an exterior wall; a membrane, wherein the membrane comprises a porous material and a non-porous material (or second porous material of substantially less pores than the porous material), and wherein the porous material is thinner than the non-porous or less porous material; and one or more heating units, wherein the one or more heating units are attached to the substrate, the one or more sources of airflow, or a combination thereof.

### Evaporative Gradient Sample Enrichment

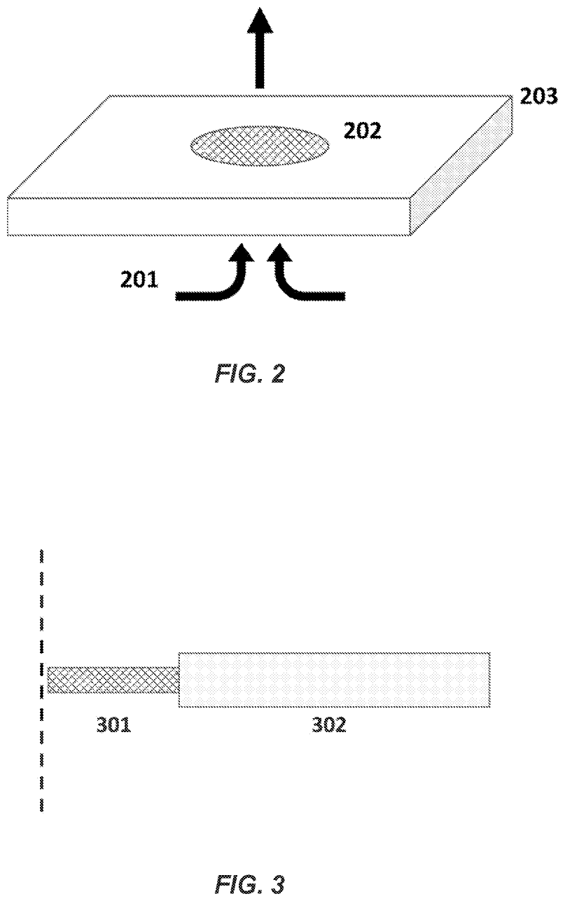

Sample enrichment can occur in a setup where an evaporative gradient is established, regardless of whether there is active or passive airflow present. One demonstration using passive airflow is shown in FIG. 2.

FIG. 2 shows a diagram of an example passive airflow sample enrichment system, in accordance with the present technology, containing both a first porous material and a second less-porous or non-porous material in a membrane, with the first porous material in the center of the membrane. In FIG. 2, the diagram shows an airflow 201 will preferentially travel through a thinner and more porous membrane 202 as compared to the bulk material 203, which is a less porous or non-porous material. The porous membrane 202 is one that can permit a flow of gas or liquid through while maintaining some absorbency to contain a portion of the solution. Some examples of porous membranes include, but are not limited to, nitrocellulose, paper, cotton, microfiber, or a combination thereof. The pores may have pores that range from less than about 0.1 mm to less than about 0.05 μm. The pores in the membrane may be evenly distributed or randomly distributed.

In some embodiments, for example, the bulk material 203 is one that does not permit a flow of gas and does not absorb the solution. Some examples of bulk materials include, but are not limited to, metal, glass, plastic, and ceramic. The bulk material 203 can be a non-porous material, but in some embodiments, the bulk material 203 can include a porous material that is substantially less porous than the porous material of the membrane 202. In some embodiments, for example, the non-porous or less porous material of the bulk material 203 may range in thickness from about 5 cm to about 0.5 mm.

Any analyte and solution containing particles which is collected in bulk material 203 will be transported towards the area of highest airflow and differential evaporation will result. This evaporation gradient can be used as a pre-enrichment step for sample collection, whereby the porous membrane 202 can be removed and inserted into a specified instrument for downstream analysis.

FIG. 3 shows a diagram depicting a cross-section of a passive airflow sample enrichment system, in accordance with the present technology, containing both a porous material and a non-porous or substantially less porous material in a membrane. The diagram of FIG. 3 shows the cross-section of the membrane with the porous membrane 301 and the bulk material 302 (e.g., non-porous or substantially less porous). While the presence of the porous membrane causes preferential airflow in ambient conditions, it also increases the evaporative gradient in active forms of airflow generation as well. This includes movement, breathing, and powered methods such as fans or vacuums to create airflow across the whole device.

### Example Results and Discussion

### Using an Airjet to Reverse the Coffee Ring Effect (CRE)

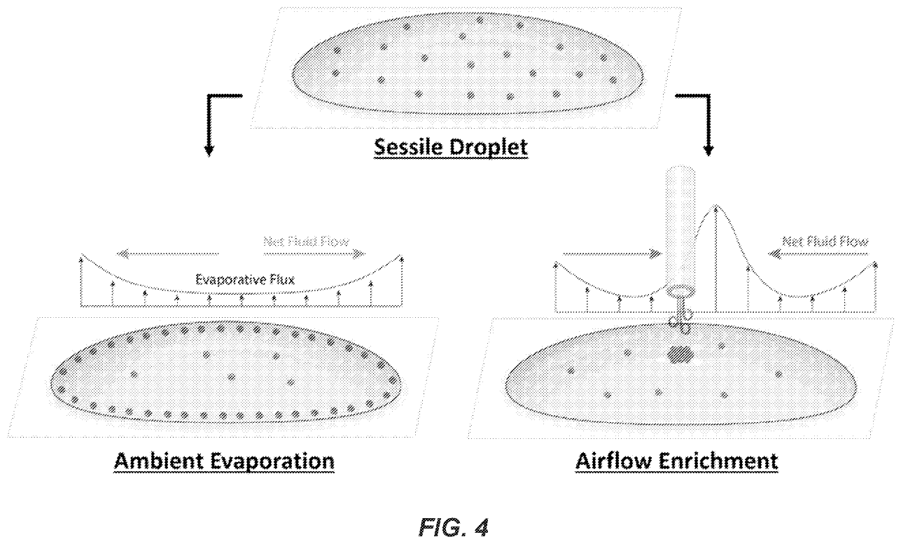

FIG. 4 shows a schematic of airjet enrichment to reverse coffee-ring flows and the comparison of the resulting deposition patterns with that of ambient evaporation.

The operating principle for airjet enrichment is depicted in FIG. 4, which illustrates the particle distribution under ambient evaporation and airjet enrichment in a sessile droplet. The development of the CRE has been attributed to the non-uniform evaporative profile across the droplet's surface. For ambient evaporation, the evaporative flux is greatest at the contact line due to the relatively larger proportion of the liquid-air interface. As a result, an evaporative gradient is established at the droplet periphery, and a radially outward flow is formed from the interior to replenish the lost fluid. This flow carries any solutes or suspended particles towards the contact line, forming a familiar ring-like pattern.

Airjet enrichment reverses this radial flow by establishing a large evaporative flux at the center of the droplet by positioning a nozzle above the droplet and directing a jet of air perpendicular to its surface. The airjet displaces the boundary layer comprised of saturated water vapor which lies directly above the liquid-air interface. Displacement of the boundary layer leads to enhanced diffusion across the interface and shifts the evaporative gradient towards the area under the airjet. In addition, as the speed of enrichment is heavily dependent on the evaporation rate and the temperature of the airjet can be used to control the overall evaporation rate.

### Suppression of Airjet-Induced Turbulence

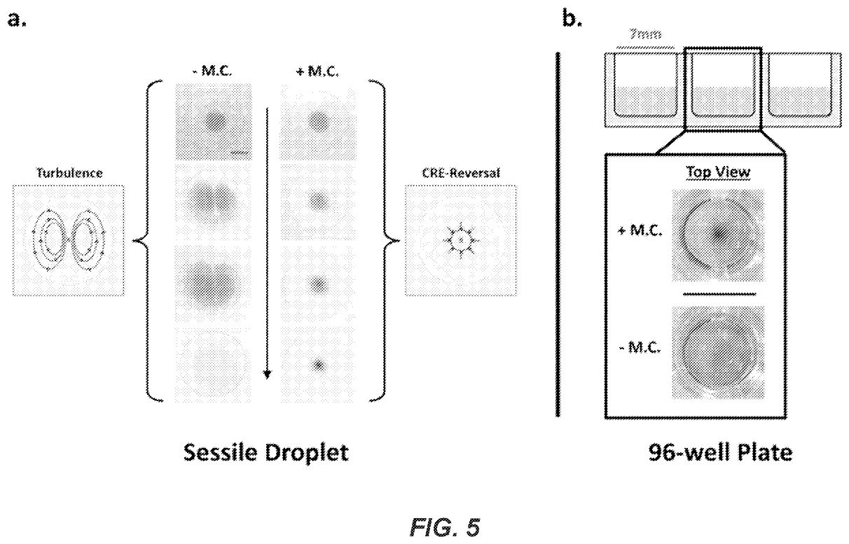

FIG. 5 shows: (a) a diagram and images of fluid behavior under an example embodiment of an airjet, in accordance with the present technology, in the absence (−M.C.) and presence (+M.C.) of the methyl cellulose surfactant in a droplet as the process progresses (from top to bottom), where the scale bar is 4 mm; and (b) a diagram and images from a 96-well plate after enrichment with (+M.C.) and without (−M.C.) methyl cellulose.

In a previous study, another airjet enrichment technique was explored using a microfluidic paper-based device, exhibiting characteristic laminar flow behavior. However, achieving laminar flow in liquids can be challenging during enrichment, as the airjet inevitably imparts shear forces on the liquid surface, leading to turbulent motion that disrupts the radial flow induced by evaporation. An additive is required to stabilize the surface and suppress streaming and turbulence to achieve the desired enrichment effect. The additive used in this study is methyl cellulose, a relatively inert and biocompatible component acting as both a surfactant and a mild thickening agent.

FIG. 5, panel a, demonstrates how the presence of methyl cellulose mitigates the airjet-induced secondary flows to achieve a condensed spot through CRE-reversal. In a 20 μL sessile droplet with a uniform yellow dye distribution, a central region of blue dye was added to visualize liquid movement. Without methyl cellulose, turbulent, vortical flow patterns quickly develop within the droplet upon initiating the airjet. This leads to increased mixing between the blue and yellow dye, eventually forming a uniformly green solution. However, by adding methyl cellulose to the droplet, the turbulent patterns are suppressed and evaporative-driven, radial flow dominates as indicated by the increasingly concentrated blue region of the dye.

Similar behavior was also observed for fluid in a 96-well plate (FIG. 5, panel b). In individual wells, 100 μL of a blue dye solution was deposited and a clear enrichment spot of concentrated dye was formed for solutions containing methyl cellulose after 8 minutes. In contrast, solutions lacking methyl cellulose had the dye evenly distributed throughout the original liquid area after the same duration. The presence of methyl cellulose is therefore critical to the enrichment process, and its addition allows enrichment to occur in various fluid containers.

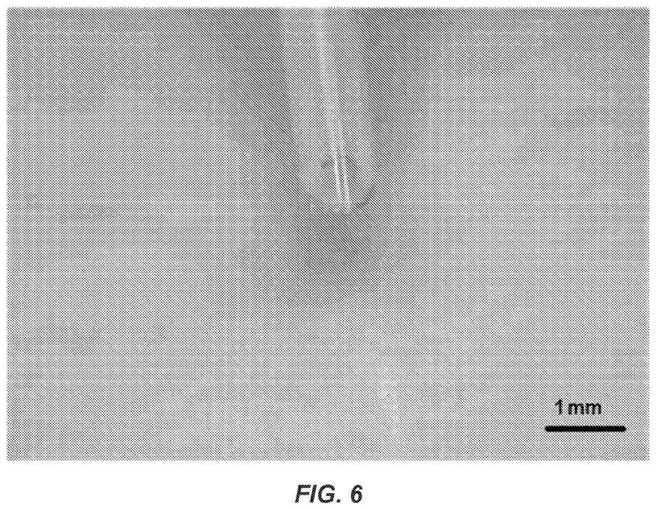

The mechanism in which methyl cellulose can suppress the airjet-induced fluid flows is attributed to its surfactant properties. Surfactants have been shown to reduce wave activity in liquids through the Gibbs-Marangoni effect. Here, the formation of capillary waves is attenuated as they would produce localized variations in surfactant density at the liquid-air interface and consequently lead to counteracting surface-tension gradients. However, not all surfactants screened during this study could completely inhibit the airjet-induced turbulence to the same degree as methyl cellulose. Therefore, additional properties are likely to play a role in the effectiveness of surface stabilization. While these example implementations focused on enrichment in aqueous solutions, it was demonstrated that the disclosed technology can also successfully perform enrichment on volatile organic solvents such as ethanol using PDMS as the surfactant (see FIG. 6). Continued exploration of surfactants and their effects on a variety of solvents will therefore yield more insight into the optimal properties required for airjet enrichment.

FIG. 6 shows an image showing an example implementation of enrichment of rhodamine in ethanol after 2 minutes. The example implementation included 20 μL of rhodamine solution deposited on a hydrophilic glass surface, and 1 μL of a 1% PDMS solution in hexane was added to the surface of the solution.

### Ubiquitous Enrichment

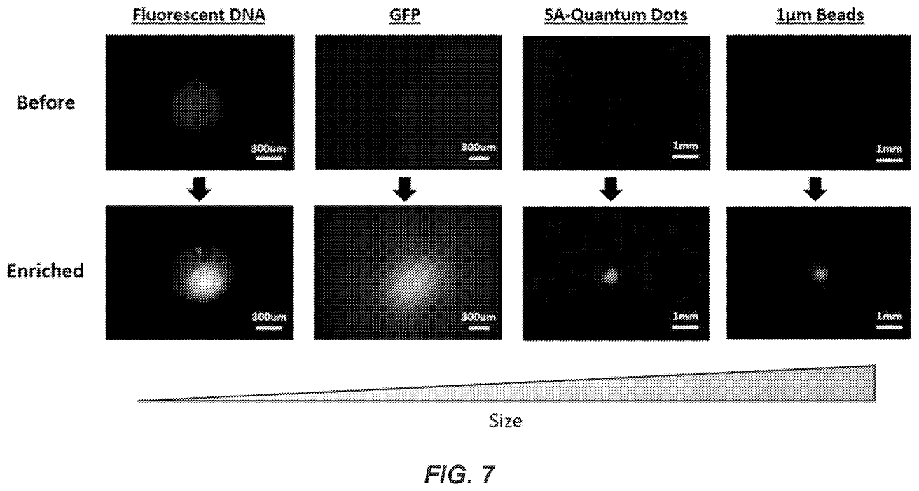

FIG. 7 shows images before and after 5 minutes of enrichment with equal exposure time for FITC-tagged DNA oligonucleotides, green fluorescent protein (GFP), 20 nm streptavidin-functionalized quantum dots, and 1 μm fluorescent beads.

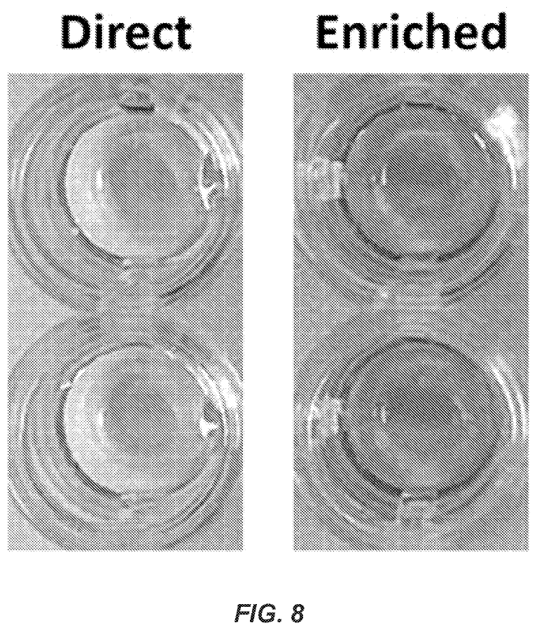

FIG. 7 shows that airjet enrichment can effectively improve detection sensitivity and concentrate various fluorescent biomacromolecules and particles, regardless of size. Solutions containing FITC-tagged DNA oligonucleotides, green fluorescent protein (GFP), 20 nm streptavidin-functionalized quantum dots, and 1 μm fluorescent beads were deposited on a hydrophilic glass slide. Methyl cellulose was added to the droplet to prevent fluid turbulence caused by airflow. A glass pipette with a 1.5 mm I.D. exit nozzle was then used to create an airjet. The gas was provided by a nitrogen tank and the strength of the airjet was controlled by a digital mass flow meter. For each solution, the fluorescent particles were concentrated to a defined spot of 300-500 μm in diameter underneath the airjet. The results in FIG. 7 demonstrate the broad applicability of this method for enriching all types of components in a liquid, independent of their physical and chemical properties. While fluorescent analytes were chosen for this study for direct detection of enrichment, successful enrichment of proteins aside from GFP was also indicated by ELISA (FIG. 8).

FIG. 8 shows images depicting a comparison of duplicate ELISA wells for direct and enriched samples after 3 minutes of Tetramethylbenzidine (TMB) development. ELISA was performed using rabbit IgG as the antigen. The direct wells contained 1 μL of a 1.3 nM solution deposited into 99 μL of PBS 1× buffer during the antigen incubation step. For the enriched solution, 100 L of 1.3 nM solution was enriched, and 1 μL of the enriched spot was withdrawn and deposited in 99 μL of PBS 1× buffer.

### Influence of a Heated Airjet on Liquid Temperatures

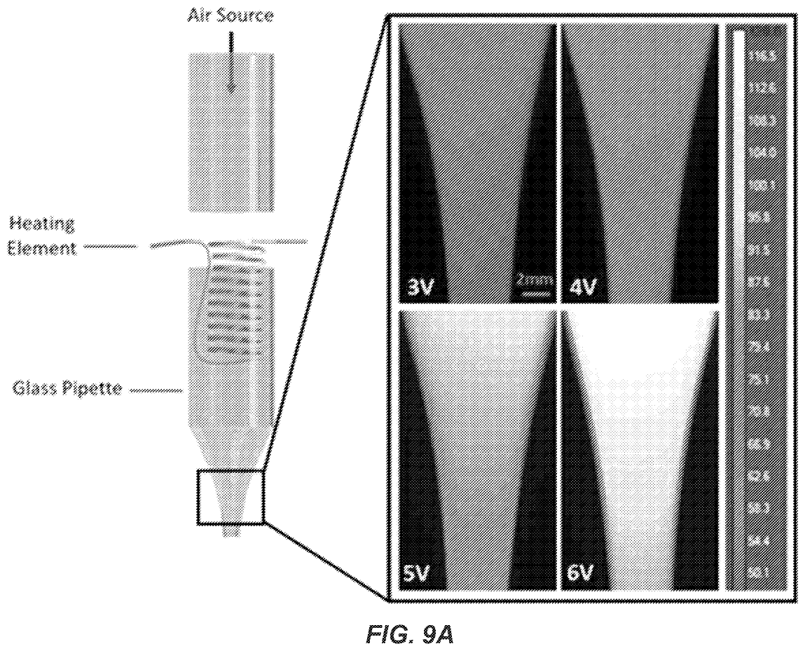

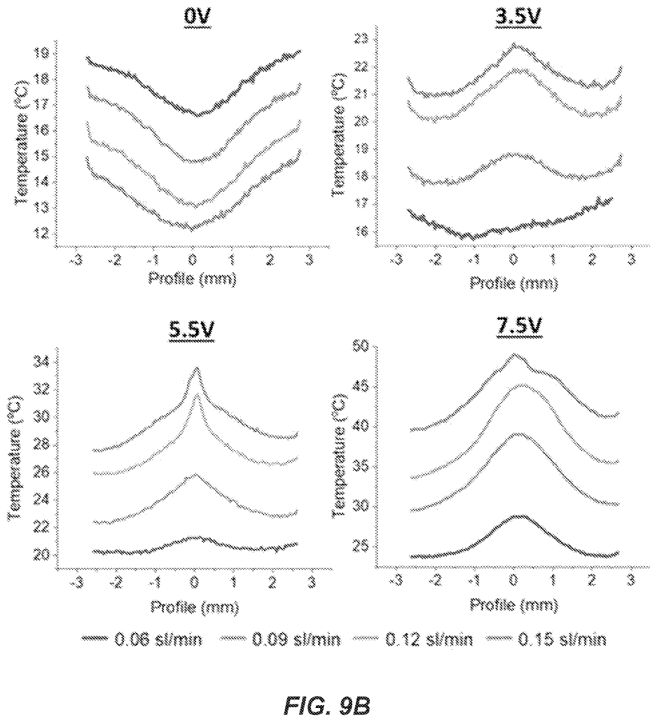

FIG. 9A shows a diagram illustrating an exemplary heated airjet (left) and associated thermal images (right) taken at specified voltages applied to the heating element; and FIG. 9B shows data plots depicting temperature profiles of the liquid surface for different voltages and airflow speeds in example implementations of the heated airjet.

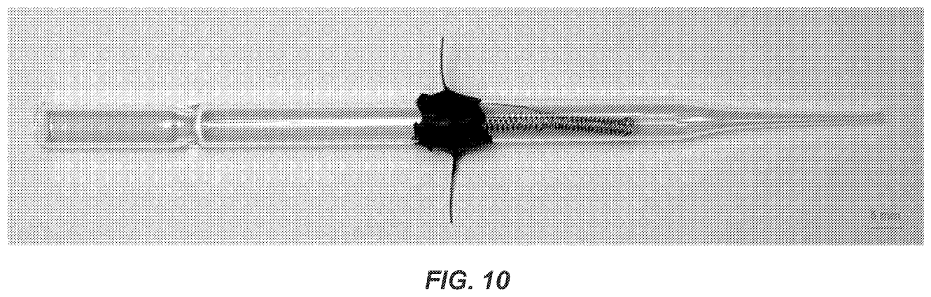

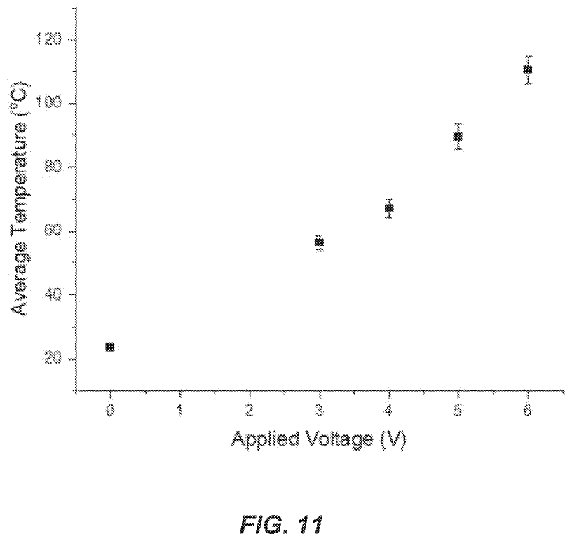

The speed of the enrichment process depends on the evaporation rate underneath the airjet. To reduce evaporation times, the airjet apparatus can include a heating element, e.g., which may comprise a nichrome coil inserted and sealed in a structure (e.g., glass pipette) of the airjet and connected to a variable voltage source (see FIG. 10). The airjet apparatus can be interfaced with an infrared camera to measure the temperature profiles near the airjet's nozzle (e.g., pipette's exit nozzle). FIG. 9A shows a data plot depicting different voltages with a constant airflow rate of 1.0 standard liter per minute (sl/m) that were used in exemplary implementations of the example embodiment of the airjet employing the heating element. A linear relationship between the voltage and mean surface temperature was established (see FIG. 11), allowing for control over the airjet temperature.

FIG. 10 shows an image of an example embodiment of the heated airjet used for enhanced sample enrichment, in accordance with the present technology. In this example, the heated airjet includes a nozzle that comprises two halves of a borosilicate glass pipette with the heating element interested in the middle. The heating element includes a 40 mm coil nichrome that is braided at the ends with wire. The glass halves are then sealed with ceramic adhesive.

FIG. 11 shows a data plot depicting average temperature vs. applied voltage for the pipette's exit nozzle for an example implementation of the sample enrichment technique using the example heated airjet of FIG. 10.

To avoid damaging bioanalytes that are vulnerable to denaturation or degradation at high temperatures, the surface temperature profile of the liquid was also measured by the infrared camera under different airjet flow rates and temperatures. FIG. 9B shows the surface temperature profiles of a liquid solution in a 96-well plate. When the nichrome heater is turned off (0V), greater airflow rates give rise to lower temperature minimums due to evaporative cooling and quicker boundary layer displacement. However, this trend is reversed upon applying voltages greater than 3.5V to the heating element, whereby faster airflow rates caused higher peak temperatures through increased levels of heat transfer. However, voltages under 5.5V did not increase the temperature above 35 °C as shown in the temperature readings in FIG. 9A, and therefore represented an upper limit to minimize degradation of bioanalytes.

For the measured temperature profile at 0V, a negative temperature gradient can give rise to inward Marangoni flows due to the temperature-dependence of surface tension in the liquid. In previous reports, Marangoni flows have been used to suppress the CRE by establishing a similar surface tension gradient through temperature differences or surfactants. By controlling the heater temperature in the air path, the technique can control the temperature profile of the liquid surface and achieve an almost isothermal profile. Since the effect of enrichment was observed under different temperature profiles, it can be concluded that the dominant effect responsible for analyte enrichment is the increased localized evaporation rate by the airjet, while the surface tension-induced Marangoni flows play only a minor role. Therefore, the primary parameters selected for the following example experiments include air flow rate and air heating to achieve high enrichment efficiency within a short time and preserve the properties of the analytes for the downstream process.

### Double-Stage Enrichment for Varying Sample Volumes

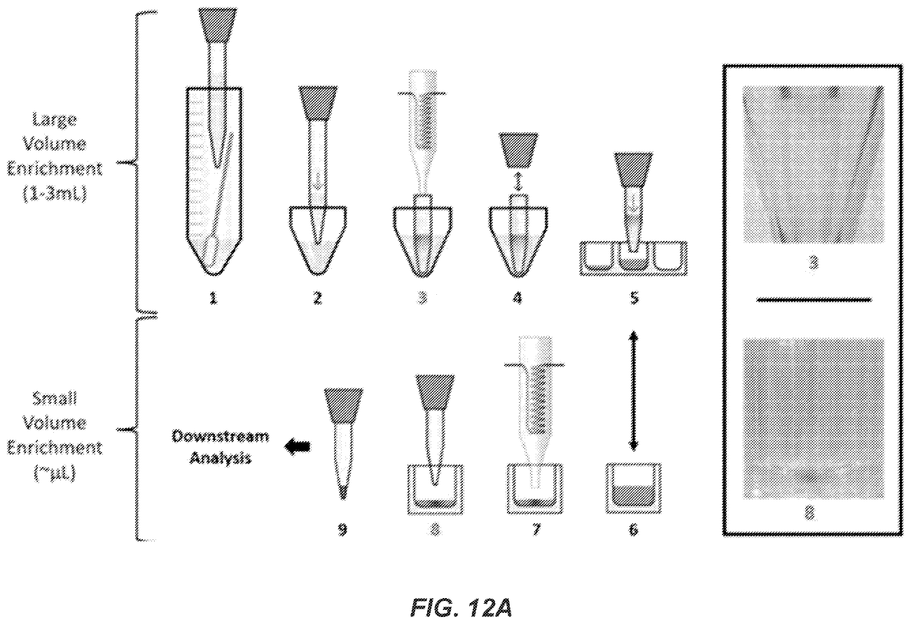

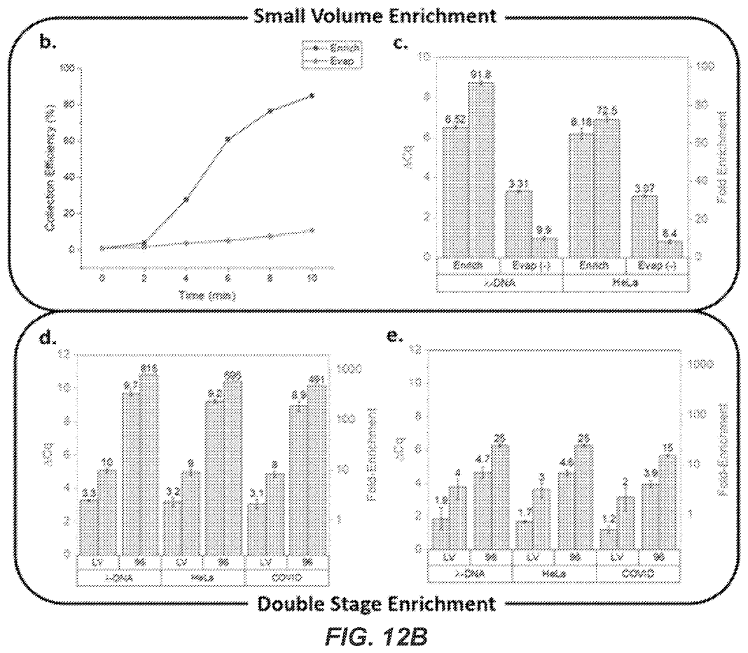

FIG. 12A shows a diagram illustrating an example embodiment of a double-stage enrichment process, with images from certain steps (i.e., steps 3 and 8). FIG. 12B shows data plots in panels b, c, d, and e, depicting: (panel b) collection efficiency vs. time for λ-DNA for airjet enrichment (Enrich) and volume reduction (Evap [−]); (panel c) comparison of ΔCq (orange) and collection efficiency (green) for λ-DNA and HeLa-S3 RNA; (panel d) cumulative ΔCq (orange) and fold-enrichment (green) for λ-DNA, HeLa-S3 RNA, and heat-inactivated SARS-COV-2 after large volume (LV) and small volume (96) enrichment; and (panel e) cumulative ΔCq and fold-enrichment for control trials using evaporative reduction only.

Incorporating airjet enrichment into the upstream steps of analyte detection can significantly improve the overall sensitivity. A model workflow was developed to demonstrate how airjet enrichment can be integrated to accommodate the fluid volumes typically used in these detection procedures (FIG. 12A). The model comprises two stages of fluid processing, each corresponding to a range of fluid volumes that can be enriched. The first stage is the large volume enrichment stage, which can enrich mesofluidic volumes (mLs), followed by the small volume enrichment stage for enriching microfluidic volumes (μLs). Each step is optimized to balance enrichment speed and effectiveness to maximize the overall concentration of analyte prior to analysis.

The large volume enrichment stage can typically process 1-3 mL of fluid by maximizing the evaporation rate. This stage has a key component-the insertion of an inner tube (FIG. 12A, step 3) where enrichment occurs. The enrichment can be visualized when the tube insert traps and localizes the enriched analytes, as indicated by the blue dye layer from the initially dilute dye solution (FIG. 12A, step 3). The tube insert acts similarly to the stabilizing properties of methyl cellulose, concentrating the enriched fluid while confining the airjet-induced shear forces. Fluid turbulence is less likely to occur due to the smaller tube dimensions. Introducing a physical barrier raises the turbulence threshold, allowing stronger airflow rates enhance the evaporation rate significantly. In addition, the inner tube acts as a collection vessel and transfer pipette for the next stage (FIG. 12A, steps 4-5).

The small-volume enrichment takes place in a 96-well plate and is designed to maximize the enrichment factor for microfluidic volumes. This stage utilizes methyl cellulose as the stabilizing additive and leads to a highly concentrated spot after enrichment (FIG. 12A, step 8). The effectiveness of airjet enrichment over conventional evaporative volume reduction is shown in FIG. 12B, panel b by tracking the collection efficiency for a λ-DNA solution over time. The collection efficiency is determined through qPCR by comparing the C_{q} values from 1 μL of the initial, unenriched sample with 1 μL of the sample undergoing enrichment. The ΔC_{q} between these two samples is used to determine the percentage of functional analytes collected at the end of the sample preparation process (i.e., the end-to-end efficiency). In this method, airjet enrichment with methyl cellulose captured 85% of λ-DNA in 1 μL volume from a 100 μL sample. In comparison, volume reduction by evaporation results in less than 10% of λ-DNA in the 1 μL transferred volume. The difference is that the airjet-enrichment method localizes the analyte underneath the nozzle for easy and efficient sample transfer, while evaporative volume reduction results in a uniformly spatial distribution within the remaining volume and suffers from the coffee-ring effect.

In addition to DNA, RNA is a frequently targeted analyte for detection but is more susceptible to degradation. Therefore, the collection efficiencies of HeLa-S3 RNA were evaluated alongside λ-DNA to determine if airjet enrichment was equally effective (FIG. 12B, panel c). Upon comparison, RNA results yielded consistently lower collection efficiencies averaging to 72%, while λ-DNA exhibited a 92% average collection efficiency. The reduced efficiency observed for RNA may be attributed to temperature-dependent auto-hydrolysis caused by the heated airjet. According to FIG. 9B, the voltage of 3.5V used during RNA enrichment would yield a mild temperature profile (between 16 and 22 °C). However, this profile measurement was taken with a filled well containing 350 μL of liquid. It is likely that during the latter stages of RNA enrichment, the reduced fluid volume of ~20 μL lowered the overall heat capacity and led to increased temperatures. Although RNA enrichment did not usually achieve the same level of collection efficiency as DNA, the collection efficiency is still far greater than the common practice of magnetic bead assays that collect less than 1% of analytes in the original sample, as elucidated previously.

To evaluate the effectiveness of the proposed workflow (FIG. 12A) in improving detection sensitivity, the large and small volume enrichment stages were combined and tested for three analytes: λ-DNA, HeLa-S3 RNA, and heat-inactivated SARS-COV-2. Cumulative ΔCq and fold-enrichment values after each enrichment stage are shown in FIG. 12B, panel d. These values reflected the performance for each step concerning the optimization of fluid reduction and enrichment effectiveness. For the large volume stage, the enrichment could consistently reduce the Cq by an average of 3.17 cycles regardless of the nucleic acid tested. More importantly, this step could evaporate off more than 1 mL of fluid and condense the analytes into 100 μL for transfer. The small volume enrichment step then significantly reduced the overall Cq by an average of 9.27 cycles within minutes. Among the analytes, λ-DNA exhibited the highest ΔCq and fold-enrichment at 9.67 and 821.23-fold, respectively, while SARS-COV-2 had the lowest values at 8.94 and 491.3-fold enrichment. RNA enrichment generally yielded lower values, consistent with the findings in FIG. 12B, panel c, and analyte loss occurred primarily during the small-volume enrichment stage.

These cumulative values can be compared to the negative control trials in FIG. 12B, panel e, where the large volume stage lacked the inner tube, and the small volume stage did not include the methyl cellulose, thereby solely relying on volume reduction to achieve enrichment. Based on this difference, the airjet enrichment stages demonstrate considerable advantages in achieving analyte enrichment.

### Selective Analyte Enrichment in High Salt Buffers

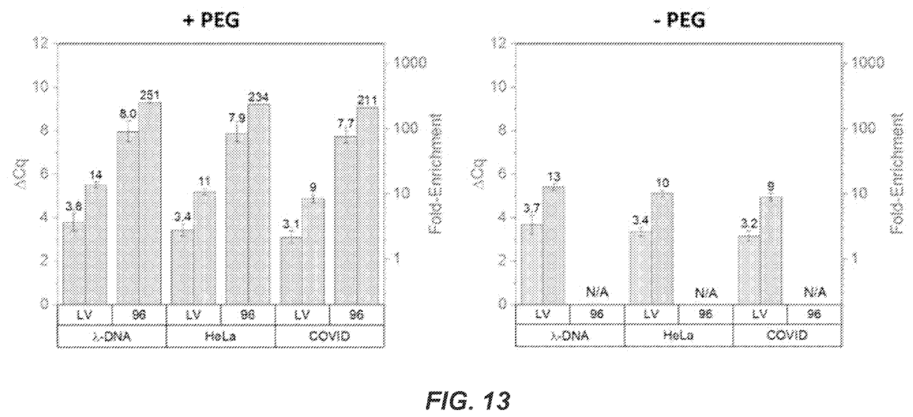

FIG. 13 shows data plots depicting a comparison of cumulative ΔCq (orange) and fold-enrichment (green) for λ-DNA, HeLa-S3 RNA, and heat-inactivated SARS-COV-2 in 0.1 mM EDTA for solutions with PEG (+PEG) (left data plot) and without PEG (−PEG) (right data plot).

While the double-stage enrichment significantly improved the overall detection sensitivity, the experiment was performed in non-buffered solutions. However, many available storage solutions contain specific salts to prevent early degradation of the analyte—often in much higher concentrations than the analyte itself. While a merit of airjet enrichment is the ability to enrich a wide range of substances, the co-enrichment of unwanted elements, particularly salt, could adversely affect downstream analyses or even damage the analytes. However, co-enrichment of specific substances can be used to create a distinct environment of limited salt solubility, and prevent salt accumulation during the analyte enrichment process. Therefore, PEG₄₀₀, a non-volatile compound in which salts have reduced solubility, was added in each sample to determine whether salt co-enrichment would decrease. The double-stage enrichment was repeated with the same analytes spiked in a solution of 5% (v/v) PEG₄₀₀ and 0.1 mM EDTA. EDTA is a chelator for divalent cations and a commonly used salt for RNA storage. However, it is also a PCR inhibitor at high concentrations as it sequesters the magnesium ions which are cofactors for polymerase activity. Co-enrichment of PEG₄₀₀ was examined to the degree by which it could reduce the ΔCq and recover the signal during downstream analysis.

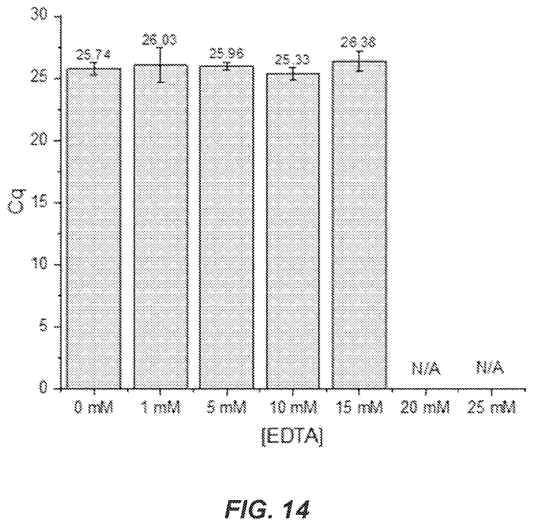

The cumulative enrichment values for λ-DNA, HeLa-S3 RNA, and heat-inactivated SARS-COV-2 in 0.1 mM EDTA are shown in (FIG. 13). The EDTA concentration threshold for qPCR functionality was determined to be 15 mM when withdrawing 1 μL of sample (FIG. 14). The effect of EDTA accumulation in downstream analysis is exemplified in the negative controls without PEG. While a qPCR signal was obtained after the large volume stage, the co-enrichment in the 96-well plate led to a high enough EDTA concentration to inhibit the PCR reaction-thereby leading to no signal. However, upon addition of a 5% PEG solution to the 96-well stage, recovery of the qPCR signal as well as enrichment was seen for all samples. Using the final λ-DNA fold-enrichment value of 250.7, it can be assumed that the same amount would also concentrate the EDTA if PEG was absent-crossing the 15 mM threshold and resulting in no qPCR signal. Therefore, the presence of a signal indicates PEG of having some desalting capabilities.

FIG. 14 shows a data plot depicting the Cq vs. EDTA concentration for a solution of λ-DNA in an example implementation of the disclosed technology. For each concentration, 1 μL from the EDTA solution was added to 9 μL of the qPCR reaction mix.

The final ΔCq values were lower in the buffered samples than those in the non-buffered samples. The addition of PEG caused this reduction, as it may mildly interfere with the function of methyl cellulose as a surfactant to suppress turbulence. Consequently, the enrichment efficiency is decreased as fluid turbulence becomes more prevalent in disrupting the formation of the enrichment spot. Therefore, adding PEG reduces the co-enrichment of salt, it also interferes with the airflow-based CRE-reversal to a certain degree. Future studies that screen for alternative compounds which minimize this trade-off would thus be beneficial.

In summary, as shown in example implementations of certain embodiments, an airjet enrichment method in accordance with the present technology can concentrate analytes to aid in sample preparation and improve downstream detection sensitivity. The method offers a direct and cost-effective approach, requiring only compressed air, a nozzle, and a surfactant to function for some embodiments. As airjet enrichment relies on reversing the coffee-ring effect, various particles and molecules can be concentrated regardless of their physical and chemical properties. The degree of enrichment was quantified using qPCR with λ-DNA, HeLa-S3 RNA, and heat-inactivated SARS-COV-2 acting as model analytes. Through CRE reversal, it was found that over 70% of λ-DNA and HeLa-S3 RNA could be localized to a 1 μL volume for transfer, a marked increase in analyte collection efficiency with prevalent methods of magnetic beads that collect ~1% analytes in the original sample. A workflow was described to adapt the enrichment method to accommodate larger volumes typically seen in nucleic acid sample preparation protocols. The modified workflow resulted in a substantial reduction in C_{q}, averaging 9.27 across all model analytes. Furthermore, adding PEG to buffered solutions allowed for selective enrichment by preventing the co-enrichment of salts and recovering a qPCR signal previously lost due to salt interference. The simplicity and effectiveness of the airjet enrichment method make it easily applicable to various common assays, indicating great potential for nucleic acid analysis in both research and clinical settings. While this study focused primarily on nucleic acids, airflow-enrichment can be applied to various chemical and biological analytes, providing enhanced detection capabilities.

### Example Implementations

### Example Reagents

Example reagents used in example implementations of the devices, systems, and methods in accordance with the present technology include the following. Methyl cellulose (M7140), PEG₄₀₀ (202398), and EDTA disodium salt (E5134) were obtained from MilliporeSigma. Carboxylate-modified 1 μm beads (T8883), streptavidin conjugated quantum dots (Q10143MP), λ-DNA (SD0011), and HeLa-S3 total RNA (AM7852) were obtained from Thermo-Fisher Scientific. Heat-inactivated SARS-COV-2 (VR-1986HK) were obtained from ATCC. FITC-labeled oligonucleotides and primers for λ-DNA, BRACAI, and NI were synthesized by Integrated DNA Technologies (IDT). For PCR analysis, iTaq Universal SYBR Green Supermix (1725120) and iTaq Universal SYBR Green One-Step Kit (1725150) were obtained from BioRad.

### Example Fabrication Techniques of Nichrome Heater

A borosilicate glass Pasteur pipette was separated into two halves. A coil from 40 mm of 28-gauge nichrome wire was made and the ends were braided with electrical wire. After the coil was inserted into the pipette half, the other half was re-sealed using a heat-resistant ceramic adhesive. The ends of the wire were connected to an adjustable DC power supply (Tekpower TP1503C) during operation.

### Airflow Evaporation on a Sessile Droplet

Glass slides were treated under UV-Ozone (Jelight Model 42) for 10 minutes. A section of cured PDMS with a 14 mm hole punched through the center was then pressed on the glass surface for a minute before removal to create a hydrophobic barrier. Solutions of the respective analyte were mixed in ultrapure H₂O containing 50 μg/mL methyl cellulose. 20 μL of the respective solution for enrichment was deposited on the hydrophilic section of the cover slip. The airjet nozzle was placed 5 mm from the droplet surface and run for the designated amount of time.

### Temperature Profiles for the Nichrome Airflow Heater

A thermal camera (FLIR A300) was used to capture thermal images for the nichrome heater and liquid in a 96-well plate. A solution of dye and 50 μg/mL methyl cellulose in Ultrapure H₂O was used as the bulk liquid and filled the well. The airflow rate and the voltages were adjusted accordingly. Three minutes were given to achieve steady state before taking the temperature profile.

### Enrichment in 96-Well Plate for DNA and RNA

The wells were treated with 200 μL of Rain-X for 5 minutes, washed twice with water, and dried prior to enrichment studies. Solutions containing dilutions of λ-DNA and 1:1000 HeLa-S3 total RNA were prepared in ultrapure H₂O. Methyl cellulose was added to bring the concentration to 50 μg/mL in addition to dilute tracking dye. For the reference sample, 1 μL of each solution was transferred to a qPCR mixture with 10 μL of total volume. The λ-DNA was enriched under the nichrome at a voltage of 3.25V and an airflow rate of 1.00 SLPM. The enrichment was continued until the liquid height reached 10% of the original height and a concentrated dye spot formed. Upon completion, 1 μL of fluid was collected from the concentrated dye spot using a micropipette and deposited into a qPCR mix. The HeLa-S3 RNA solution was performed similarly, except the airflow rate was reduced to 0.75 SLPM when the liquid height reached 25% of the original.

### Double-Stage Enrichment

To create the large volume container, the tops of a 15 mL falcon tube were cut off at the 2.5 mL mark and the bottom half was saved. A circular piece of foil containing an 8 mm hole in the center was then adhered to the top of the tube. Solutions of diluted λ-DNA, or HeLa-S3 RNA, or heat-inactivated COVID-19 particles were made in ultrapure H₂O containing a small amount of tracking dye. In the cut tube, 2 mL of each solution was added. The ends of a pipette tip were trimmed, and the tip was placed in the hole. The nichrome nozzle was placed in the interior of the pipette tip and set at a flow of 2.0 SLPM at a voltage of 7.5V. The enrichment was performed until 1 mL evaporated off and a distinct layer of concentrated dye formed. The enrichment in the 96-well plate was performed as previously described, adding 1 μL of 5 mg/mL methyl cellulose to the well to achieve spot enrichment. After enrichment in the large volume and 96-well containers, 1 μL of the enriched sample was transferred to a qPCR reaction and compared with 1 μL of reference sample from the initial nucleic acid solution. Negatives were run simply based on evaporation, where the inner tube was not present for the large volume enrichment, and no methyl cellulose was present in the 96-well enrichment.

### Buffered Enrichment Using PEG

Similar solutions of λ-DNA, or 1:1000 HeLa-S3 RNA, or 1:500 of heat-inactivated COVID-19 particles were made in 0.1 mM EDTA with a small amount of blue tracking dye. The respective solutions were transferred to the large-volume container and evaporated similarly to the original series enrichment. After forming of the enriched layer, 100 μL of the layer was collected and deposited in a 96-well plate. In each individual well, 1 μL of 5 mg/mL methyl cellulose and 5 μL of PEG₄₀₀ were mixed to form a homogenous solution. The enrichment was run according to the previous 96-well plate protocol.

### Examples

In some embodiments in accordance with the present technology (example 1), a method for sample enrichment includes distributing a plurality of particles in a solution under ambient evaporation; and applying a flow of gas through one or more airjets to increase concentration of the plurality of particles in the solution.

Example 2 includes the method of example 1 or any of examples 1-16, wherein the flow of gas applied through the one or more airjets reverses radial flow by establishing a large evaporative flux at a center of a droplet of the solution.

Example 3 includes the method of example 1 or any of examples 1-16, wherein the flow of gas applied through the one or more airjets displaces a boundary layer comprised of saturated water vapor which lies directly above a liquid-air interface.

Example 4 includes the method of example 3 or any of examples 1-16, wherein displacement of the boundary layer leads to enhanced diffusion across the liquid-air interface and shifts an evaporative gradient towards an area under the one or more airjets.

Example 5 includes the method of any of examples 1-4 or any of examples 1-16, further comprising controlling an overall evaporation rate based at least on controlling temperature of the flow of gas through the one or more airjets.

Example 6 includes the method of any of examples 1-5 or any of examples 1-16, further comprising controlling an overall evaporation rate based at least on controlling temperature of the solution.

Example 7 includes the method of any of examples 1-6 or any of examples 1-16, wherein the flow of gas applied through the one or more airjets includes positioning a nozzle above a droplet of the solution and directing a jet of air perpendicular to its surface.

Example 8 includes the method of any of examples 1-7 or any of examples 1-16, wherein the gas includes at least one of nitrogen, argon, air, or a combination thereof.

Example 9 includes the method of any of examples 1-16, wherein the droplet is a sessile droplet.

Example 10 includes the method of any of examples 1-9 or any of examples 1-16, further comprising adding a surfactant to the solution before the applying the flow of gas through the one or more airjets.

Example 11 includes the method of example 10 or any of examples 1-16, wherein the surfactant includes one or more of methyl cellulose, polydimethylsiloxane, or sodium dodecyl sulfate.

Example 12 includes the method of any of examples 1-11 or any of examples 1-16, wherein the one or more airjets are set up in parallel.

Example 13 includes the method of any of examples 1-11 or any of examples 1-16, wherein the one or more airjets are set up in an array.

Example 14 includes the method of any of examples 1-11 or any of examples 1-16, wherein the one or more airjets are aimed at the same location.

Example 15 includes the method of any of examples 1-11 or any of examples 1-16, wherein the one or more airjets are aimed at more than one location.

Example 16 includes the method of any of examples 1-15, wherein the plurality of particles include one or more of nucleic acid molecules, polynucleotides, viruses, dissolved components, suspended components, or a combination thereof.

In some embodiments in accordance with the present technology (example 17), a method for sample enrichment includes distributing a plurality of particles in a solution under ambient evaporation; applying the solution to a porous membrane; and applying a flow of gas through one or more airjets to increase concentration of the plurality of particles in the solution.

Example 18 includes the method of example 17 or any of examples 17-27, wherein the porous membrane includes one or more of paper, cotton, microfiber, bamboo, nitrocellulose, or a combination thereof.

Example 19 includes the method of any of examples 17-18 or any of examples 17-27, further comprising controlling an overall evaporation rate based at least on controlling temperature of the flow of gas through the one or more airjets.

Example 20 includes the method of any of examples 17-19 or any of examples 17-27, further comprising controlling an overall evaporation rate based at least on controlling temperature of the solution.

Example 21 includes the method of any of examples 17-20 or any of examples 17-27, wherein the flow of gas applied through the one or more airjets includes positioning a nozzle above the solution and directing a jet of air perpendicular to its surface.

Example 22 includes the method of any of examples 17-21 or any of examples 17-27, wherein the gas includes at least one of nitrogen, argon, air, or a combination thereof.

Example 23 includes the method of any of examples 17-22 or any of examples 17-27, wherein the one or more airjets are set up in parallel.

Example 24 includes the method of any of examples 17-22 or any of examples 17-27, wherein the one or more airjets are set up in an array.

Example 25 includes the method of any of examples 17-22 or any of examples 17-27, wherein the one or more airjets are aimed at the same location.

Example 26 includes the method of any of examples 17-22 or any of examples 17-27, wherein the one or more airjets are aimed at more than one location.

Example 27 includes the method of any of examples 17-26, wherein the plurality of particles include one or more of nucleic acid molecules, polynucleotides, viruses, dissolved components, suspended components, or a combination thereof.

In some embodiments in accordance with the present technology (example 28), a method for sample enrichment includes distributing a plurality of particles in a solution under ambient evaporation; applying the solution to a membrane, the membrane comprising (i) a first material comprising a porous material and (ii) a second material comprising a non-porous material or a second porous material having substantially less pores than the porous material of the first material; and creating a differential evaporation rate through airflow from one or more sources between one or more locations of the membrane to increase the concentration of the plurality of particles or dissolved or suspended components in the solution at locations on the membrane where the evaporation rate of the solution is greater.

Example 29 includes the method of example 28 or any of examples 28-40, wherein the porous material of the first material includes one or more of paper, cotton, microfiber, bamboo, nitrocellulose, or a combination thereof.

Example 30 includes the method of example 28 or any of examples 28-40, wherein the porous material of the first material is a cellulose-based material.

Example 31 includes the method of any of examples 28-30 or any of examples 28-40, wherein the porous material of the first material is thinner than the non-porous material or second porous material of the second material.

Example 32 includes the method of any of examples 28-31 or any of examples 28-40, wherein the airflow from the one or more sources between the one or more locations of the membrane are passive airflow sources.

Example 33 includes the method of any of examples 28-32 or any of examples 28-40, wherein the airflow from the one or more sources between the one or more locations of the membrane are active airflow sources.

Example 34 includes the method of any of examples 28-33 or any of examples 28-40, further comprising controlling an overall evaporation rate based at least on controlling temperature of the airflow.

Example 35 includes the method of any of examples 28-34 or any of examples 28-40, wherein a gas in the airflow includes at least one of nitrogen, argon, air, or a combination thereof.

Example 36 includes the method of any of examples 28-35 or any of examples 28-40, wherein the one or more sources of the airflow are set up in parallel.

Example 37 includes the method of any of examples 28-35 or any of examples 28-40, wherein the one or more sources of the airflow are set up in an array.

Example 38 includes the method of any of examples 28-35 or any of examples 28-40, wherein the one or more sources of the airflow are aimed at the same location.

Example 39 includes the method of any of examples 28-35 or any of examples 28-40, wherein the one or more sources of the airflow are aimed at more than one location.

Example 40 includes the method of any of examples 28-39, wherein the plurality of particles include one or more of nucleic acid molecules, polynucleotides, viruses, dissolved components, suspended components, or a combination thereof.

In some embodiments in accordance with the present technology (example 41), a device or system for sample enrichment includes an airjet-induced liquid phase enrichment technique operable to implement the method of any of examples 1-16, the method of any of examples 17-27, or the method of any of examples 28-40.

In some embodiments in accordance with the present technology (example 42), a device for sample enrichment includes one or more sources of airflow; a membrane; and a heating unit.

Example 43 includes the device of example 42 or any of examples 42-55, wherein the one or more sources of airflow further comprise an additional heating unit.

Example 44 includes the device of example 42 or 43 or any of examples 42-55, wherein the one or more sources of airflow include at least one airjet, wherein the at least one airjet comprises a nozzle structure comprising two regions with the heating element interested in a middle zone of the two regions.

Example 45 includes the device of example 44 or any of examples 42-55, wherein the heating element includes a coil nichrome that is braided at its ends with wire leading outside the nozzle structure of the airjet.

Example 46 includes the device of any of examples 42-45 or any of examples 42-55, wherein the membrane is porous.

Example 47 includes the device of any of examples 42-45 or any of examples 42-55, wherein the membrane includes one or more of paper, cotton, microfiber, bamboo, nitrocellulose, or a combination thereof.

Example 48 includes the device of example 42 or 43 or any of examples 42-55, wherein the membrane comprises a first porous material and a second material that is a non-porous material or a less porous material than the first porous material.

Example 49 includes the device of example 48 or any of examples 42-55, wherein the first porous material includes at least one of paper, cotton, microfiber, bamboo, nitrocellulose, or a combination thereof.

Example 50 includes the device of any of examples 42-49 or any of examples 42-55, wherein the one or more sources of the airflow are set up in parallel.

Example 51 includes the device of any of examples 42-50 or any of examples 42-55, wherein the one or more sources of the airflow are set up in an array.

Example 52 includes the device of any of examples 42-50 or any of examples 42-55, wherein the one or more sources of the airflow are aimed at the same location.

Example 53 includes the device of any of examples 42-50 or any of examples 42-55, wherein the one or more sources of the airflow are aimed at more than one location.

Example 54 includes the device of any of examples 42-53 or any of examples 42-55, wherein the one or more sources of the airflow are passive.

Example 55 includes the device of any of examples 42-53 or any of examples 42-54, wherein the one or more sources of the airflow are active.

Example 55 includes the device of any of examples 42-55, wherein the device is configured to implement the method of any of examples 1-16, the method of any of examples 17-27, or the method of any of examples 28-40.

In some embodiments in accordance with the present technology (example 56), a device for sample enrichment includes one or more sources of airflow; a substrate, wherein the substrate is a non-porous material, and wherein the substrate includes a base and an exterior wall; a membrane, wherein the membrane comprises a first material comprising a porous material and a second material comprising a non-porous material or a less porous material than the first porous material, and wherein the first porous material is thinner than the second material; and one or more heating units, wherein the one or more heating units are attached to the substrate, the one or more sources of airflow, or a combination thereof.

Example 57 includes the device of example 56, wherein the device is configured to implement the method of any of examples 1-16, the method of any of examples 17-27, or the method of any of examples 28-40.

### Conclusion

A “nucleic acid molecule” or “polynucleotide” refers to a polymeric compound including covalently linked nucleotides comprising natural subunits (e.g., purine or pyrimidine bases). Purine bases include adenine, and guanine, and pyrimidine bases including uracil, thymine, and cytosine. Nucleic acid molecules include polyribonucleic acid (RNA), polydeoxyribonucleic acid (DNA), which includes cDNA, genomic DNA, and synthetic DNA, either of which may be single or double-stranded. A nucleic acid molecule encoding an amino acid sequence includes all nucleotide sequences that encode the same amino acid sequence.

The term “about” as used herein in the context of a number refers to a range centered on that number and spanning 10% less than that number and 15% more than that number. The term “about” used in the context of a range refers to an extended range spanning 10% less than that the lowest number listed in the range and 15% more than the greatest number listed in the range.

Throughout this disclosure, any concentration range, percentage range, ratio range, or integer range is to be understood to include the value of any integer within the recited range and, when appropriate, fractions thereof (such as one tenth and one hundredth of an integer), unless otherwise indicated. Also, any number range of this disclosure relating to any physical feature, such as polymer subunits, size, or thickness, are to be understood to include any integer within the recited range, unless otherwise indicated. Throughout this disclosure, numerical ranges are inclusive of their recited endpoints, unless specifically stated otherwise.

The terminology used in the description is intended to be interpreted in its broadest reasonable manner, even though it is being used in conjunction with a detailed description of identified embodiments.

It is intended that the specification, together with the drawings, be considered exemplary only, where exemplary means an example. As used herein, the singular forms “a”, “an” and “the” may be construed in the singular and in the plural forms as well, as the context indicates. Additionally, the use of “or” may be construed as an exclusive “or” or as “and/or,” as the context indicates.

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing unit” or “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

1. A method for sample enrichment, comprising:
distributing a plurality of particles in a solution under ambient evaporation; and
applying a flow of gas through one or more airjets to increase concentration of the plurality of particles in the solution.

2. The method of claim 1, wherein the flow of gas applied through the one or more airjets reverses radial flow by establishing a large evaporative flux at a center of a droplet of the solution.

3. The method of claim 1, wherein the flow of gas applied through the one or more airjets displaces a boundary layer comprised of saturated water vapor which lies directly above a liquid-air interface.

4. The method of claim 3, wherein displacement of the boundary layer leads to enhanced diffusion across the liquid-air interface and shifts an evaporative gradient towards an area under the one or more airjets.

5. The method of claim 1, further comprising controlling an overall evaporation rate based at least on controlling temperature of the flow of gas through the one or more airjets and/or controlling temperature of the solution.

6. (canceled)

7. The method of claim 1, wherein the flow of gas applied through the one or more airjets includes positioning a nozzle above a droplet of the solution and directing a jet of air perpendicular to its surface.

8. The method of claim 1, wherein the gas includes at least one of nitrogen, argon, air, or a combination thereof.

9. The method of claim 7, wherein the droplet is a sessile droplet.

10. The method of claim 1, further comprising adding a surfactant to the solution before the applying the flow of gas through the one or more airjets.

11. The method of claim 10, wherein the surfactant includes one or more of methyl cellulose, polydimethylsiloxane, or sodium dodecyl sulfate.

12. The method of claim 1,
wherein the one or more airjets are set up in parallel or set up in an array; and or
wherein the one or more airjets are aimed at a same location or aimed at more than one location.

13.-15. (canceled)

16. The method of claim 1, wherein the plurality of particles include one or more of nucleic acid molecules, polynucleotides, viruses, dissolved components, suspended components, or a combination thereof.

17. A method for sample enrichment, comprising:
distributing a plurality of particles in a solution under ambient evaporation;
applying the solution to a porous membrane; and
applying a flow of gas through one or more airjets to increase concentration of the plurality of particles in the solution.

18. The method of claim 17, wherein the porous membrane includes one or more of paper, cotton, microfiber, bamboo, nitrocellulose, or a combination thereof.

19. The method of claim 17, further comprising controlling an overall evaporation rate based at least on controlling temperature of the flow of gas through the one or more airjets and/or controlling temperature of the solution.

20. The method of claim 17, further comprising controlling an overall evaporation rate based at least on controlling temperature of the solution.

21. The method of claim 17, wherein the flow of gas applied through the one or more airjets includes positioning a nozzle above the solution and directing a jet of air perpendicular to its surface.

22. The method of claim 17, wherein the gas includes at least one of nitrogen, argon, air, or a combination thereof.

23. The method of claim 17,
wherein the one or more airjets are set up in parallel or set up in an array; and or
wherein the one or more airjets are aimed at a same location or aimed at more than one location.

24.-27. (canceled)

28.-40. (canceled)

41. (canceled)

42.-56. (canceled)

57. A device for sample enrichment, comprising:
one or more sources of airflow;
a substrate, wherein the substrate is a non-porous material, and wherein the substrate includes a base and an exterior wall;
a membrane, wherein the membrane comprises a first material comprising a porous material and a second material comprising a non-porous material or a less porous material than the first porous material, and wherein the first porous material is thinner than the second material; and
one or more heating units, wherein the one or more heating units are attached to the substrate, the one or more sources of airflow, or a combination thereof.

58. (canceled)
